(19) United States
(12) Patent Application Publication
(10) Pub. No.: US 2017/0325426 A1
(43) Pub. Date: Nov. 16, 2017

(54) A Method and Device for Remote Monitoring of Animals

(71) Applicant: Herd Moonitor Ltd., Raanana (IL)

(72) Inventors: Arieh BROSH, Bat Shlomo (IL); Sinay GOLDBERG, Raanana (IL)

(21) Appl. No.: 15/519,873

(22) PCT Filed: Jul. 29, 2015
(86) PCT No.: PCT/IL2015/000036

§ 371 Date: Apr. 18, 2017

Related U.S. Application Data
(60) Provisional application No. 62/065,666, filed on Oct. 18, 2014.

(51) Int. Cl. A01K 29/00; A61B 5/00; A01K 11/00; A61B 5/08; A61B 5/11; A61B 5/113

(52) U.S. Cl. CPC A01K 29/005; A61B 5/1123; A61B 5/113; A01K 11/008; A61B 5/0816; A61B 5/0022; A61B 2503/40

(57) ABSTRACT

A method, device and system are provided for remotely monitoring one or more parameters associated with a grazing animal. The method comprises the steps of: mounting a collar comprising sensor onto a grazing animal; detecting by said sensor movements of the grazing animal or lack thereof; classifying the detected movements into a group of pre-defined activity classes; and based detected movements and their classification into at least one of the pre-defined activity classes, determining at least one of the parameters being monitored.

### TECHNICAL FIELD

The present disclosure relates to the field of monitoring animals behavior and in particularly, monitoring animals behavior of grazing animals.

### BACKGROUND

Optimal herd management is based on adjustment of cows' needs for production and reproduction by diet nutritional quality and availability and by fast response to sickness, epidemic and extreme climate events. Most of the large herds in the world graze on large unmonitored areas without any communication network coverage. Consequently, herds' production and weaning rate, are far from its' potential.

Keeping track of herd health status and reproduction state have been of major interests in the cattle industry. Some systems have been provided during the years, which were mounted on all the animals in the herd, but this was done mainly for confined treated livestock, which their movements can be easily monitored and which graze at nearby locations.

U.S. Pat. No. 4,618,861 describes a system wherein every time an animal comes in the immediate vicinity of the sensor, a count representing the detected movements of the animal is read, when the animals are supposed to be milked twice per twenty-four hours and, consequently, to go twice a day to the milking parlour, where a sensor for reading out the counts is installed. These counts are averaged over a number of days where the average value thus obtained, can be compared every new count. A new count which is sufficiently above this average value, indicates female estrus. When a new count is sufficiently below this average value, this will be an indication that the animal has an injured leg or is ill, or at least has contracted a disease. In this manner, by means of such an animal activity meter, it is possible to monitor health events in general and diseases, injuries and the estrus events in particular.

EP 743,043 discloses a system for measuring the activity of an animal. By this solution, the animal's movements can be continuously counted over a pre-determined period of time of e.g. five minutes, a quarter of an hour, etc., and when this pre-determined period of time has elapsed, the count is recorded in a memory means. When the animal comes in the vicinity of a sensor, a series of counts can be extracted. In this manner, the activity pattern may accurately be updated. When an animal is lying and ruminating, the count will be very low. During this period of rest, it will not be necessary to measure the animal activity frequently. In such a situation, it is possible to provide the animal activity meter with a sensor for measuring the distance to the ground and for inciting a signal indicating that the animal is lying, standing or walking. In the memory means the moments can then be recorded when the animal lies down and rises.

However, there is a long felt need for a system and method that provide information on the condition of individual animals and/or of herds of animals, which graze at an open remote location and obviously do not access a pre-defined point (a point where a sensor of the prior art solutions may be installed).

### SUMMARY OF THE DISCLOSURE

One object of the disclosure is to provide a device, a system and a method that enable retrieving information that relate to parameters characterizing the status of individual animals that are remotely located at open pastures (e.g. large grazing areas), such as for example energy balance (nutrition state), health and reproductive events of the grazing animals by using a behavior activities analysis.

It is another object of the present disclosure is to provide a device, a system and a method that enable retrieving information that relate to parameters characterizing the status of a herd of livestock at a remote open pastures.

It is another object of the present disclosure is to provide a method for carrying out a behavior activities analysis of the grazing animals.

Other objects of the present invention will become apparent from the following description.

According to a first embodiment there is provided a method for remotely monitoring one or more parameters associated with a grazing animal (e.g. a free grazing animal), wherein the method comprises the steps of:
(i) mounting a collar comprising sensor onto a grazing animal;
(ii) detecting by said sensor movements of the grazing animal or lack thereof;
(iii) classifying the detected movements into a group of pre-defined activity classes. For example, the classification of these groups may be based on data collected by the sensor (such as an accelerometer) mounted onto the grazing animal and used to determine a frequency at which a certain activity took place by the respective animal. In addition or in the alternative, it may be based on the time of the day when the activities took place, groups that are based on the daily periods of time which the activities took place, frequency at which the same one or more activities took place during a pre-defined period of time, etc.);
(iv) based on detected movements and their classification into at least one of the pre-defined activity classes, determining at least one of the parameters being monitored.

In accordance with another embodiment, the group of pre-defined activity classes comprises one or more of the following members: resting, grazing, walking and mastication while resting (which mainly represent rumination).

By yet another embodiment, the step of classifying the detected movements into a group of pre-defined activity classes, further comprises utilizing data that relates to energy and/or frequency of the animal's head and/or neck movements during grazing, during browsing for forage, and/or for detecting the animal's respiration rate.

According to still another embodiment, the sensor is a member of a group that consists of one or more inertial sensors, an image capturing device and a combination thereof.

In accordance with another embodiment, the method provided further comprising a step of transmitting data that relates to movements of the animal being monitored.

According to another embodiment, the method further comprising a step of transmitting data that relates to a current location of the animal being monitored.

By yet another embodiment, the step of classifying the detected movements into a group of pre-defined activity classes, is based on an algorithm applied onto data retrieved from the inertial sensor.

According to still another embodiment, the method further comprises a step of remotely monitoring one or more parameters associated with a plurality of free grazing animals, wherein the one or more parameters is determined based on data collected from detected movements of each of the plurality of the free grazing animals.

In accordance with another aspect of the disclosure, there is provided a device mountable of an animal (e.g. in a form of a collar) for remotely monitoring parameters associated with a free grazing animal, the device comprising:
a. at least one sensor adapted to detect movements of the free grazing animal;
b. at least one processor configured to classify the detected movements into a group of pre-defined activity classes; and
c. a transmitter configured to transmit data associated with the detected movements.

By another embodiment the at least one processor is further adapted to determine a frequency of the animal's body and/or head and/or neck movements for detection of heat stress.

According to another embodiment, the at least one processor is further adapted to determine respiration rate, (e.g. for detecting heat stress).

According to still another embodiment, the sensor is a member of a group that consists of one or more inertial sensors, an image capturing device and a combination thereof.

In accordance with another embodiment, the device further comprising a storage (e.g. a memory means) configured to store data that relates to movements of the animal being monitored.

According to still another aspect of the disclosure, there is provided a system for remotely monitoring parameters associated with a grazing animal, the system comprising:

a plurality of devices described above for remotely monitoring parameters associated with a grazing animal;

a receiver operative to receive data transmitted from the transmitters of each of the plurality of the remotely monitoring devices;

a central processor configured to process data received at the receiver and determine therefrom said one or more parameters based on data collected from detected movements of each of the plurality of the grazing animals.

According to another embodiment of this aspect, the system further comprises a transmitter configured to transmit communications to at least one of the plurality of the remotely monitoring devices, and wherein the at least one of the plurality of the remotely monitoring devices, is further provided with a receiver configured to receive these communications.

In accordance with another aspect there is provided a non-transitory computer readable medium storing a computer program for performing a set of instructions to be executed by one or more computer processors, the computer program is adapted to perform the method provided herein.

### BRIEF DESCRIPTION OF THE DRAWINGS

For a more complete understanding of the present invention, reference is now made to the following detailed description taken in conjunction with the accompanying drawings wherein:

FIGS. 1A to 1C—demonstrate embodiments of the method provided for classifying cows' activities by the algorithm provided by the present invention vs. the cows' speed as determined by respective GPS devices.

FIG. 2—illustrates a device (a collar) to be mounted on an animal for the purpose of detecting its movements and establish therefrom its activities; and

FIG. 3—illustrates a schematic view of an example system for monitoring health events, reproductive status and nutrition state of a herd of free grazing animals.

### DETAILED DESCRIPTION

In this disclosure, the term “comprising” is intended to have an open-ended meaning so that when a first element is stated as comprising a second element, the first element may also include one or more other elements that are not necessarily identified or described herein, or recited in the claims.

In the following description, for the purposes of explanation, numerous specific details are set forth in order to provide a better understanding of the present invention by way of examples. It should be apparent, however, that the present invention may be practiced without these specific details.

In the following examples, readily available MEMS based accelerometers were used for determining activity and health related events of livestock (e.g. cows), by using and efficient and drift resilient cow activity classification algorithm for analyzing 3-axis accelerometer data.

Since power for computing is scares in solar powered device, an efficient algorithm was established to analyze the inertial sensor data. This algorithm read the inertial sensor output for predefined period of time and outputs a single number which can is correlated to the energy of such activity.

In the tests conducted, data was collected by using a data logger comprised in a neck collar mounted at the dorsal side of the cow's neck. The algorithm numerical output was compared to visual observation of the cow's activity and cows speed measurement by GPS.

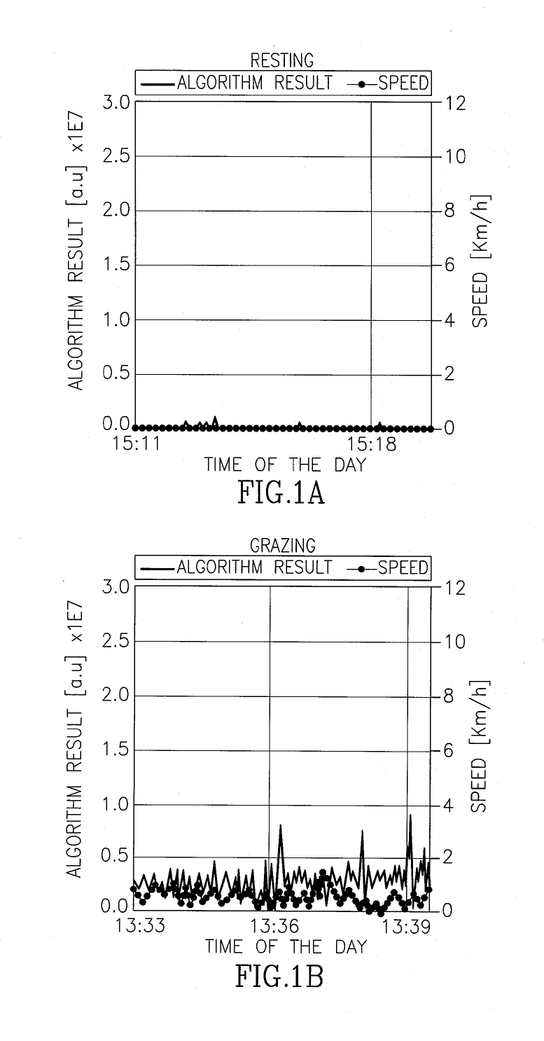

FIGS. 1A to 1C demonstrate typical outputs of the algorithm applied according to embodiments of the present invention showing results of GPS-based speed for various cow's activities. The results shown in these three Figs. present results achieved by using that algorithm (dark lines) and the speed determined by using GPS readings (gray lines).

The three graphs show distinct differentiation between algorithm outputs at different activities. At resting (FIG. 1A), the algorithm output is smaller than 600,000 a.u. (where “a.u.” is used to denote arbitrary units for quantifying differences between results obtained under various activities. At grazing, (FIG. 1B), the algorithm output is between 600,000 and 4,500,000 a.u., with few spikes relating to walking between grass patches, while at walking (FIG. 1C) the results obtained by the algorithm used, are greater than 4,500,000 a.u.

The cow collar that was used as the data logger comprised the following parts:

1) a sealed plastic box mounted at the top of the neck, and contained the electronics.
2) straps from the plastic box mounted around the cow's neck to a balance weight.
3) balancing weights to keep the collar in an upper position on the cow's neck.

Within the logger collar there were an inertial measurement, RF transmitter, solar harvesting electronics and panels. The validation resulting data of comparison the Applicant's algorithm to speed measurements is shown in FIG. 1, the validation of its comparison to direct cows activities observation is presented in Table 1. Table 1 data showed that there is no substantial overlapping between the algorithm calculation′ range of the three activities (rest, graze, walk).

TABLE 1
Cow's activities direct observations and their
classification (thousands) by applying the algorithm of the
present invention

| Activity observation | Average | SE | max | min | N |
|---|---|---|---|---|---|
| Rest | 268 | 10 | 449 | 176 | 51 |
| Rest Stand | 300 | 12 | 566 | 239 | 32 |
| Rest Lay Down | 279 | 13 | 535 | 179 | 40 |
| Rest while ruminate | 392 | 07 | 618 | 239 | 113 |
| Grazing Woody Area | 1,536 | 92 | 3,685 | 750 | 50 |
| Grazing Herbage | 1,865 | 59 | 3,926 | 1,277 | 51 |
| Walking | 8,664 | 870 | 36,268 | 4,734 | 36 |

### Wherein:

SE is the standard error;
max is a maximal value for the respective activity;
min is a minimal value for the respective activity; and
N is a number of observations.

Based on the method described hereinabove that was used to determine different cow's activities, the next step is to determine the current energy balance (nutritional state) of the cows. For example, heat production (HP), a term which relates to energy expenditure of the cow that represents its balance (MEI=HP+RE), where MEI is metabolizable energy intake, the available energy for animals metabolic needs, and RE is the recovered energy, i.e. the energy retained within the animal body+the energy content of the produced milk.

Various parameters may be used in the process of evaluating the status of an individual animal and/or of a herd to which a plurality of animals belong. Among these parameters there are the following ones:

1) Daily changes in the energy balance status of an individual cow: This parameter may be calculated from changes in the individual cow's daily grazing time. There is a significant variation between animals' efficiency of using the diet for maintenance and production. Consequently changes in the individual daily grazing time represent changes to the individual energy balance, for example reduction in daily grazing time of an individual cow from 8 hours to 5 hours indicates a significant reduction in daily intake and MEI;
2) Herd energy balance. Value of the herd energy balance which is determined from the herd average daily grazing time. The daily herd's average grazing time will used to calculate herd energy balance parameters.
3) Quality of the grazed (the consumed feed) herbage (metabolizable energy concentration, ME) can be calculated from knowing herd's average daily grazing time.
4) Health events of individual cows: This parameter may be retrieved from individual reduction of both, daily grazing time and daily walking time (e.g. increasing resting time), compared with the previous days, provided that the average daily grazing time and the average walking distance of the herd to which the individual cow belongs, remain essentially constant, unless another behavior (like coming calving) is expected.
5) Health events in the herds (epidemics): This parameter may be derived from results showing that from day to day more and more animals exhibit a certain abnormal behavior (e.g. resting time) while the rest of the monitored herd behavior of daily grazing time and daily walking time still remains similar to that exhibited before.
6) Heat detection of cows (estrus): A cow in estrus walks more, eats less and rests less. Consequently, when individual cow behavior is compared with its behavior in previous days, if a cow is in estrus; it will walk for a longer time and will rest (and may graze) for less time, which leads to an increased ratio of daily walking time to daily resting time.
7) Conception date (pregnancy) and calving date of each individual cow: Cows are in a cycle of estrus every 19 to 22 days. Cow pregnancy duration is almost constant (280-285 days, breed depended). Cow has to be at a specific energy balance to begin estrus and to complete it in conception. A decrease in daily resting time and an increase in daily walking time indicate that the cow is in estrus. When the above behavior is not repeated in an interval of about 19 to 23 days and the energy balance of the herd (indicating by average daily grazing time of the cows that are not in estrus) is not decreased substantially during those 19 to 23 days, it means that the cow has successfully conceived in the former estrus cycle date and consequently the expected date of calving would be 280-285 days from the last heat detection date. Identifying short period (up to about 15 days or less) between two events of heats is an indication of a problem in the ovaries (cysts).

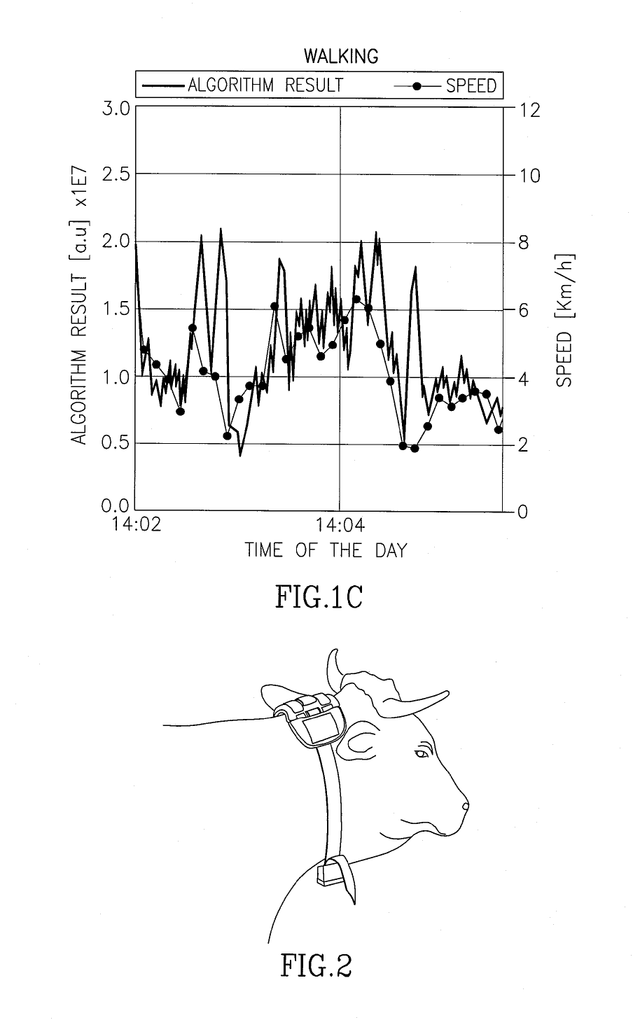

Reference is now made to FIG. 2 which illustrates in a non-limiting manner a device to be mounted on an animal for the purpose of detecting its movements and establish therefrom its activities. The device comprising: at least one first module comprising a sensor which is adapted to detect movements of the grazing animal, and a processor operative to identify and classify the detected movements into animal activities. This first module is configured to be mounted on the animal. At least one second module adapted to transmit data generated by the processor of the first module, and at least one third module comprising (i) solar panels for recharging the electronic components of the two other modules and (ii) power management. As will be appreciated, this third module may be replaced by one or more batteries the can supply the power needed to operate the first two modules described above.

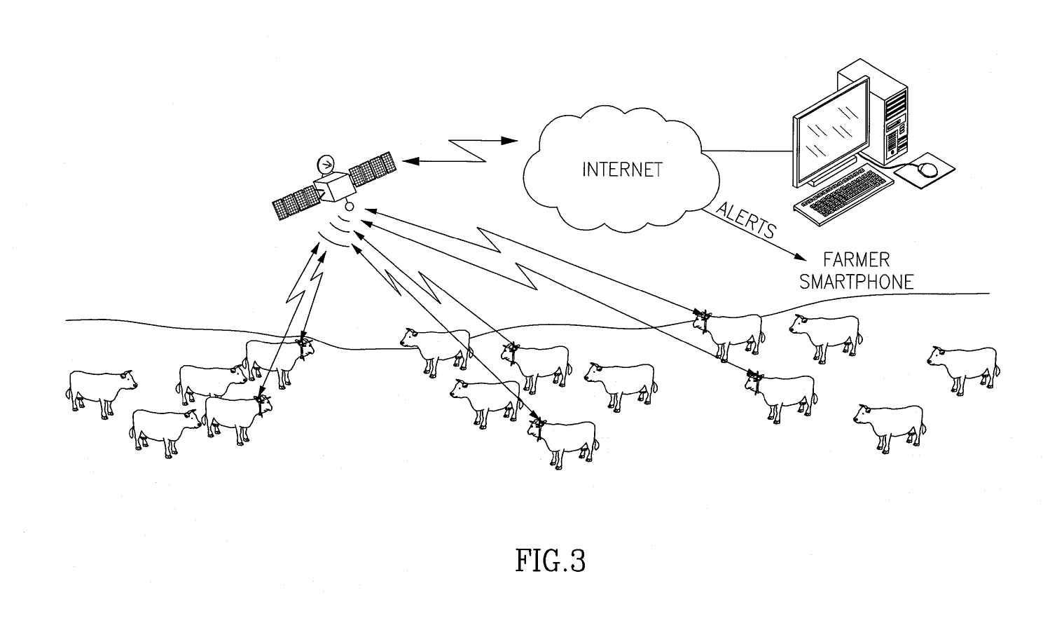

FIG. 3 illustrates a non-limiting example of a system for monitoring animal herd health status comprising a plurality of devices and at least one central processing unit (e.g. a computer readable medium (CRM)) storing instructions to enable receiving data transmitted by second modules from each of the plurality of devices of the animals belonging to that herd. The CRM is operative to (i) determine each animal health status from the data received from a respective one of the plurality of devices that it mounted on that animal; and (ii) determine the animal herd health status from data received from a plurality of devices (a plurality which may be smaller than the plurality of the devices that are mounted on animals that belong to that herd).

In addition, as may be seen in this FIG. while each device is configured to transmit data (by its second module) that relates to the activities of the animal it is mounted on, the device (e.g. the second module) is also configured to receive transmissions (e.g. by a satellite via which communications are exchanged between the CRM and the animal's devices) generated from the central processing entity (e.g. the CRM) and conveyed towards the individual animals. The latter communications (which are transmitted to the individual animal's device) are used for example to affect changes in collecting and/or analyzing data that will eventually be transmitted from the animal. The communications from the central processing entity towards the animals' devices may be in a way of broadcasting (e.g. for all the animals belonging to the herd to receive the same communication), or of a unicasting type (e.g. to one or more devices associated with certain respective individual animals).

In some embodiments of the current invention, the first module is adapted to detect information selected from a group that comprises sum of: daily resting time (lying down and standing), daily grazing time and daily walking (traveled without grazing) time, number of head movement, amplitude of head movement, frequency of head movement, number of head movement while grazing, number of head movement while grazing and browsing forage, frequency of head movements, frequency of head movement while grazing, frequency of head movement while grazing and browsing forage, travelling distance for a predetermined time interval, geographical location at a predetermined time and any combination thereof.

The direct information that will be gathered by the system may be for example: daily activities time that will be classified into 3 or 4 categories: lying down and standing (resting), grazing, walking (walking without grazing) and number and/or frequency of head movement for grazed and for browse forage. Further information that may also be collected is daily mastication duration (mainly rumination), traveling distance when grazing, when walking without grazing, daily total and animals' geographical location at predefined time of the day.

While the preferred embodiment and various alternative embodiments of the invention have been disclosed and described in detail herein, it may be apparent to those skilled in the art that various changes in form and detail may be made therein without departing from the spirit and scope thereof.

1. A method for remotely monitoring one or more parameters associated with a free grazing animal, wherein the method comprises the steps of:
(i) mounting a collar comprising sensor onto a grazing animal;
(ii) detecting by said sensor movements of the grazing animal or lack thereof;
(iii) classifying the detected movements into a group of pre-defined activity classes; and
(iv) based on detected movements and their classification into at least one of the pre-defined activity classes, determining at least one of the parameters being monitored.

2. The method of claim 1, wherein the group of pre-defined activity classes comprises one or more of the following members: resting, rumination while resting, grazing and walking.

3. The method of claim 1, wherein the step of classifying the detected movements into a group of pre-defined activity classes, further comprises utilizing data that relates to frequency of the animal's head and/or neck movements during grazing, during browsing for forage, and/or for detecting the animal's respiration rate.

4. The method of claim 1, wherein the at least one parameters is a member of a group that consists of: daily changes in the energy balance status of individual free grazing animals, herd energy balance, quality of the grazed herbage, health events of individual grazing animals, detection of estrus of free grazing animals and conception date and expected calving date of individual grazing animals

5. The method of claim 1, wherein the sensor is a member of a group that consists of one or more inertial sensor, an image capturing device and a combination thereof.

6. The method of claim 1, further comprising a step of storing data that relates to movements of the animal being monitored.

7. The method of claim 1, further comprising a step of transmitting data that relates to movements of the animal being monitored.

8. The method of claim 1, further comprising a step of transmitting data that relates to a current location of the animal being monitored.

9. The method of claim 1, wherein the step of classifying the detected movements into a group of pre-defined activity classes, is based on an energy-correlation algorithm applied onto data retrieved from the sensor.

10. The method of claim 1, further comprising a step of remotely monitoring one or more parameters associated with a plurality of free grazing animals, wherein said one or more parameters is determined based on data collected from detected movements of each of the plurality of the free grazing animals.

11. A device for remotely monitoring parameters associated with a grazing animal, said device comprising:
a. at least one sensor adapted to detect movements of the grazing animal;
b. at least one processor configured to classify the detected movements into a group of pre-defined activity classes; and
c. a transmitter configured to transmit data associated with the detected movements.

12. The device of claim 11, wherein the group of pre-defined activity classes comprises one or more of the following members: resting, grazing and walking.

13. The device of claim 11, wherein said at least one processor is further configured to determine frequency of the animal's head movements for grazing and for browsing for forage, and utilize that information in classifying the detected movements into a group of pre-defined activity classes.

14. The device of claim 11, wherein the sensor is a member of a group that consists of one or more inertial sensors, an image capturing device and a combination thereof.

15. The device of claim 11, further comprising a storage configured to store data that relates to movements of the animal being monitored.

16. The device of claim 11, further comprising a GPS configured to identify a current location of the animal being monitored.

17. The device of claim 11, further comprising a receiver configured to receive transmissions generated by a central processing entity for affecting changes in collecting and/or analyzing data by said device.

18. A system for remotely monitoring parameters associated with a grazing animal, said system comprising:
a plurality of devices of claim 11 for remotely monitoring parameters associated with a grazing animal;
a receiver operative to receive data transmitted from the transmitters of each of the plurality of the remotely monitoring devices;
a central processor configured to process data received at the receiver and determine therefrom said one or more parameters based on data collected from detected movements of each of the plurality of the free grazing animals.

19. The system of claim 18, further comprising a transmitter configured to transmit communications to at least one of the plurality of the remotely monitoring devices, and where said at least one of the plurality of the remotely monitoring devices, is further provided with a receiver configured to receive said communications.

20. A non-transitory computer readable medium storing a computer program for performing a set of instructions to be executed by one or more computer processors, the computer program is adapted to perform the method of claim 1.
